(19) United States
(12) Patent Application Publication
(10) Pub. No.: US 2003/0113784 A1
(43) Pub. Date: Jun. 19, 2003

(54) Regulated expression of recombinant DNA

(76) Inventors: Jaime Flores-Riveros, Thousand Oaks, CA (US); Jean Adamus, Guilford, CT (US); Grazia Ambrosini, New Haven, CT (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/306,482

(22) Filed: Nov. 27, 2002

Related U.S. Application Data
(60) Provisional application No. 60/333,970, filed on Nov. 29, 2001.

(51) Int. Cl. C12Q 1/68; C12Q 1/66; C07H 21/04; C12N 9/00; C12N 9/02; C12N 9/24; C12P 21/02; C12N 5/06

(52) U.S. Cl. 435/6; 435/8; 435/69.1; 435/320.1; 435/325; 435/183; 435/189; 435/200; 536/23.2

(57) ABSTRACT

This invention relates to regulatable recombinant expression constructs that provide regulated gene expression of mammalian genes.

This application claims priority to U.S. provisional patent application Serial No. 60/333,970, filed Nov. 29, 2001, the disclosure of which is incorporated by reference herein in its entirety.

### BACKGROUND OF THE INVENTION

1. Field of the Invention

This invention discloses recombinant expression constructs that provide regulated gene expression of mammalian genes. Specifically, the invention provides such regulated gene expression of mammalian, most preferably human genes. In particular, the invention provides recombinant expression constructs, recombinant promoters, recombinant cells and methods of making and using said constructs and cells to produce mammalian proteins in a regulated, most preferably in an inducible manner.

2. Background of the Related Art

Molecular biological and genetic engineering techniques permit isolation and expression of almost any known gene. A variety of robust expression systems exist, and some have been packaged into commercially-available kits. Such expression systems include systems for constitutive as well as regulated, most typically inducible, gene expression.

Recombinant promoters comprising expression regulatory elements are known in the art.

Hu & Davidson, 1987, Cell 48: 555-566 demonstrate that the bacterial lac operator-repressor pair is functional in mammalian cells.

Recombinant, engineered lac repressor constructs, particularly comprising nuclear localization signals, are known in the art.

European Patent Application, Publication No. 332416A2 discloses the use of a prokaryotic repressor protein to regulate gene expression in a mammalian cell.

Brown et al., 1987, Cell 49: 603-612 showed that the lac repressor can regulate expression from a hybrid SV40 early promoter in animal cells.

Figge et al., 1988, Cell 52: 713-722 showed that the E. coli lac repressor could inhibit expression of chloramphenicol acetyl transferase.

Deuschle et al., 1989, Proc. Natl. Acad. Sci. USA 86: 5400-5404 disclose that foreign genes can have regulated expression in mammalian cells under the control of coliphage T3 RNA polymerase and lac repressor.

Biard et al., 1992, Biochim. Biophys. Acta 1130: 68-74 showed that the E. coli lac operon could be used to express in human cells.

Syroid et al., 1992, Molec. Cell. Biol. 12: 4271-4278 demonstrate regulated expression of a mammalian nonsense suppressor tRNA gene in vivo and in vitro using the lac operator/repressor.

Hannan et al., 1993, Gene 130: 233-239 disclosed an engineered PGK promoter and lac operator-repressor system for the regulation of gene expression in mammalian cells.

Recombinant expression constructs have been used to demonstrate regulated gene expression.

U.S. Pat. No. 5,169,760 disclosed a lac operon sequence in which the catabolite activating protein (CAP) portion of the promoter was deleted, and the claim was not explicitly limited to expression in bacterial cells.

Edamatsu et al., 1997, Gene 187: 289-294 disclose an inducible high-level expression vector for mammalian cells, pEF-LAC carrying human elongation factor 1α promoter and lac operator.

The lac repressor and the lactose operon are well-known in the art. Jacob and Monod, 1961, “Genetic regulatory mechanisms in the synthesis of proteins,” J. Molec. Biol. 3: 318-356; the lac operon is discussed in detail in Lewin, 1974, GENE EXPRESSION-1, J. Wiley & Sons: N.Y.,-pp. 272-309.

Genetically engineered lac repressor genes and gene products are known in the art.

Labow et al., 1990, Molec. Cell. Biol. 10: 3343-3356 disclose an engineered lac repressor that was converted into an allosterically regulated transcriptional activator in mammalian cells.

U.S. Pat. No. 5,622,840 disclosed a lac repressor proteins having specific mutations in the amino acid sequence thereof that render the protein temperature sensitive.

Although a number of regulatable constructs utilizing the lac repressor are known in the art, there is no generally useful, regulatable promoter that can be used to reliably express desired genes of interest. There is thus a need in the art for regulatable mammalian promoter constructs that can be used in vitro, ex vivo and in vivo to induce or de-repress expression of desired genes in response to easily-administered small molecule regulators.

### SUMMARY OF THE INVENTION

This invention provides regulatable mammalian promoter constructs and recombinant expression constructs encoding desired genes that can be used in vitro, ex vivo and in vivo to induce or de-repress expression of desired genes in response to easily-administered small molecule regulators.

In a first aspect, the invention provides a recombinant expression construct encoding a gene operably linked to a genetically-engineered promoter comprising transcriptional control elements that are responsive to a small molecule regulator. In these constructs, gene expression is regulated by contacting a cell containing the recombinant expression construct with the small molecule regulator. In preferred embodiments, the promoter comprises at least two DNA operator sequences inserted proximal to a transcription initiation site in said promoter that mediate regulated expression from the promoter. In preferred embodiments, at least one of the DNA operator sequences is arranged in a negative or reverse orientation to the direction of transcription. In preferred embodiments, the promoter is a tissue-specific promoter. In additional preferred embodiments, the promoter is a synthetic promoter, or a synthetic, tissue-specific-promoter. In preferred embodiments, the gene is a reporter gene. In alternative preferred embodiments, the gene is a eukaryotic gene, more preferably a mammalian gene and most preferably a human gene, the regulated expression of which provides for optimal production of the gene product in vitro or in vivo. In alternative embodiments, the gene, encodes an antigen, most preferably a tumor antigen, a viral antigen, a bacterial antigen or a protozoal antigen. In yet further embodiments, the construct comprises an enhancer sequence, more preferably a tissue-specific enhancer sequence, where in most preferred embodiments the enhancer is active in a cell type specific for a tissue-specific promoter. In additional preferred embodiments, an intron is inserted before the translational start of the expressed gene.

In a second aspect the invention provides a recombinant cell, most preferably a recombinant mammalian cell, that comprises a recombinant expression construct of the invention. In certain embodiments, the recombinant cell further comprises a second recombinant expression construct that encodes a regulatory protein that regulates expression from the genetically-engineered promoter. In some of these embodiments, the second recombinant expression construct is covalently linked to and a part of the recombinant expression construct encoding the desired gene. In yet alternative embodiments, the recombinant cell expresses an endogenous regulatory protein that regulates expression from the genetically-engineered promoter of the recombinant expression constructs of the invention. In further alternative embodiments, the recombinant cell comprises a tissue ex vivo or in vivo.

In a third aspect the invention provides a regulatory protein and a nucleic acid encoding said regulatory protein, wherein the protein regulates expression from the genetically-engineered promoter of the recombinant expression construct.

In preferred embodiments, the regulatory protein further comprises a nuclear localization signal sequence. In a fourth aspect the invention provides methods for producing regulated expression of a gene in a mammalian cell. The inventive methods comprise the steps of introducing a recombinant expression construct of the invention into a cell that further comprises a transcriptional regulatory protein that mediates transcriptional regulation. Transcriptional regulation by the regulatory protein is accomplished by contacting the cell with an effective amount of the small molecule regulator wherein the regulatory protein recognizes said operator sequences and regulated transcription thereby.

In a fifth aspect the invention provides methods for producing regulated expression of a gene in an animal. In this aspect the method comprises the steps of introducing into the animal a recombinant expression construct of the invention and a second construct encoding a transcriptional regulatory protein that mediates transcriptional regulation of the promoter by recognizing said operator sequences. Regulated expression is achieved by administering to the animal an effective amount of the small molecule regulator to the animal.

In certain embodiments of the methods and constructs of the inanition are provided methods for treating an animal with a disease, wherein a gene is either under-expressed or a mutant gene is expressed. In this inventive method, a recombinant expression construct of the invention is introduced into cells of the animal and expression of the gene induced using a small molecule regulator.

In a sixth aspect are provided methods for producing a recombinant promoter element under transcriptional control of a transcriptional regulator protein. In this embodiment, the method comprises the step of digesting DNA encoding a promoter with a restriction enzyme having a recognition site proximal to an mRNA transcription initiation site. The digested promoter DNA is mixed with a molar excess of a double-stranded oligonucleotide encoding the transcription regulatory sequence recognized by said transcriptional regulator protein. The mixture is ligated under conditions wherein the ligatable ends of the double-stranded oligonucleotide are in excess, wherein at least two copies of the double-stranded oligonucleotide are incorporated at the restriction digestion site. The recombinant expression construct so obtained is used according to the methods of the invention to produce a gene product of interest. In additional embodiments, the promoter is a synthetic promoter, wherein oligonucleotides encoding the synthetic promoter can be introduced into a existing promoter or can be the basis for producing a synthetic promoter de novo.

Specific preferred embodiments of the present invention will become evident from the following more detailed description of certain preferred embodiments and the claims.

### BRIEF DESCRIPTION OF THE DRAWINGS

FIG. 1 is an autoradiogram of an immunoblot of cell fraction extracts from untransfected HEK293 cells and HEK293 cells that have been stably transfected with a LacR-encoding plasmid (5-D4). The arrow shows the position of LacR protein.

FIGS. 2A-2D are photographs of immunostaining of 5-D4 cells stained with preimmune serum and rhodamine-conjugated secondary antibody (FIG. 2A), anti-LacR antibody and rhodamine-conjugated secondary antibody (FIG. 2B), or corresponding staining with DAPI (FIG. 2C for preimmune serum and FIG. 2D for anti-LacR antibodies).

FIG. 3 is an autoradiogram of an electrophoretic mobility shift assay of nuclear extracts from 5-D4 cells and untransfected HEK 293 cells incubated with ³²P-labeled double-stranded oligonucleotides having the lac operator sequence. Lanes 5-7 show competition of LacR-lac operator complex formation with increasing concentration of unlabeled lac operator double-stranded oligonucleotides, and Lanes 8-10 show uninhibited complex formation in the presence of control, unrelated double-stranded oligonucleotide.

FIG. 4 is a diagram of the physical map of the SV40 early promoter region (present in the pSVP-galactosidase (pSV-β-gal) plasmid), which was used to produce the lac operator-containing recombinant expression constructs of the invention.

FIGS. 5A and 5B is a diagram illustrating the structure of the SV40-derived recombinant promoters of the invention comprising lac operator sequences. FIG. 5C is a diagram illustrating the structure of further SV40-derived recombinant promoters of the invention comprising lac operator sequences. FIG. 5D is a diagram of a physical map of the Op4 plasmid and derivatives thereof.

FIG. 6A is a histogram showing isopropylthiogalactoside (IPTG) induction of β-galactosidase expression in cells expressing the LacR and containing lac operator inserted pSVβ-gal plasmids as described in Example 2.

FIG. 6B is a histogram showing beta-galactosidase activity of Op4 and derivatives thereof in the presence and absence of IPTG in 5-D4 cells.

FIG. 7A is a histogram showing IPTG induction of beta-galactosidase expression in 5-D4 cells expressing the LacR and containing lac operator inserted pSVβ-gal plasmids as described in Example 2.

FIG. 7B is a histogram showing beta-galactosidase activity in C2C12 myoblasts transiently transfected with both the LacR and the lac operators in the presence and absence of IPTG.

FIGS. 8A and 8B are diagrams of pSVβ-gal and Op4 promoter constructs, showing the partial 5′ UTR to the start of translation, including the confirmed transcriptional start sites in pSV-β-gal (FIG. 8A) and Op4 (FIG. 8B). The hatched lines in each bar under the diagrams represent the predicted size of a fragment produced by RNase protection assay in HEK293 cells expressing protein from these promoters. FIG. 8C is a photograph of gel electrophoretic analysis of the products of an RNase protection assay of the Op4 transcriptional start site, showing the predicted fragment sizes. 1×10⁵ cpm labeled hybridized RNA probe was loaded in each lane. Lane 1) RNA century marker 2) Mouse β-actin control (expected protected size 250 bases) 3) HEK-293 cells only 4) HEK-293/pSV-β-galactosidase and 5) HEK-293/Op4.

FIG. 9 sets forth the sequence of muscle-specific enhancer elements from the human myosin light chain 1 (MLC1) locus.

FIG. 10 is a histogram showing IPTG induction of beta-galactosidase expression in in vitro differentiated C2C12 myoblasts containing LacR plus Op4-6 and Op8 plasmids and their derivatives as described in Example 2.

FIG. 11 is a histogram showing the time course of IPTG induction of beta-galactosidase expression in LacR-expressing C2C12 myoblasts containing Lac-operator inserted pSVβ-gal plasmids as described in Example 2.

FIGS. 12A and 12B are diagrams of the constructs Op4hskiME and Op4SPiME. FIG. 12A is a schematic of Op4/hskiME containing the 523 bp full-length endogenous human skeletal alpha-actin (hskA) promoter in the reporter construct. FIG. 12B is a schematic of Op4/SPiME, with the synthetic promoter containing myogenic-specific enhancer binding sites MEF-1 and MEF-2, transcriptional element TEF-1, and the serum response elements (SRE). This SP is cloned upstream of the partial human skeletal a-actin promoter. The SV40 intron and MLC1 muscle enhancer are also shown.

FIG. 13A is a histogram showing beta-galactosidase activity in the absence or presence of IPTG in HeLa or C2C12 myoblasts transiently transfected with the LacR and containing different recombinant promoter constructs of the invention in the luciferase reporter vector pGL3 basic (Promega).

FIG. 13B is a histogram showing beta-galactosidase activity in the absence or presence of IPTG in C2C12 myotubes. LacR and different recombinant promoters in the luciferase reporter vector pGL3 basic were transiently transfected into myoblasts, then differentiated for 14 days in vitro.

FIGS. 14A and 14B are histograms showing results of experiments demonstrating IPTG induction of beta-galactosidase expression in C2C12 myoblasts (FIG. 14A) and in myotubes differentiated for 14 days in vitro (FIG. 14B).

### DETAILED DESCRIPTION OF THE PREFERRED EMBODIMENTS

The present invention provides genetically-engineered promoter elements, and recombinant expression constructs comprising said promoter elements operably linked to at least one gene wherein expression of said gene from the genetically-engineered promoter is regulatable by small molecule regulators. The invention also provides recombinant cells comprising said recombinant expression constructs, as well as cells comprising heterologous regulatory proteins and nucleic acid encoding said regulatory proteins, wherein the genetically-engineered promoter elements are regulated by the regulatory proteins.

The terms “expression construct” and “recombinant expression construct” will be understood to describe genetically-engineered nucleic acid sequences encoding at a minimum an origin of replication, a selectable marker and a gene or polypeptide-encoding nucleic acid of interest to be expressed in a recipient host cell.

As used herein, the term “regulatable” is intended to encompass at least two alternative embodiments. In the first embodiment, the regulatory protein binds to or otherwise interacts with regulatory DNA sequences in the genetically-engineered promoter element, so that as a consequence transcription from the promoter is inhibited in the absence of the small molecule regulator. In this embodiment the small molecule regulator interrupts or interferes with binding or other interaction between the promoter elements and the regulatory protein, so that transcription is de-repressed or activated in the presence of the small molecule regulator. In an alternative second embodiment, the regulatory protein fails to bind to or otherwise interacts with regulatory DNA sequences in the genetically-engineered promoter element in the absence of the small molecule regulator, wherein said binding or interaction is necessary for transcription from the promoter or otherwise activates transcription therefrom. In these embodiments, binding to other interaction between the small molecule regulator and the regulatory protein causes, enhances or facilitates binding of the regulatory protein to the promoter elements, so as a consequence transcription from the promoter is de-repressed or activated in the presence of the small molecule regulator.

As used herein, the term “regulatory protein” is intended to encompass any protein that activates, represses or modulates transcription from a promoter by binding to or otherwise interacting with genetic operator elements, most preferably DNA sequences, contained in or in functional proximity to the promoter. Examples of such regulatory proteins include bacterial proteins such as the lacI repressor from the lactose operon of E. coli (as described more fully in Muller-Hill, 1996, THE LAC OPERON: A SHORT HISTORY OF A GENETIC PARADIGM, deGruyter Publishing: New York, incorporated by reference herein), catabolite activator protein (CAP), tryptophan operon proteins trpR and trpS, lambda phase cro and cI genes, as well as mammalian regulatory proteins such as NFicB, NFI, cyclic AMP responsive element binding protein (CREB), MyoD I, homeobox transcription factors, Sp1, the oncogenes fos and jun, Mep-1, GATA-1, Isl-1, LFB1, NFAT, Pit-1, OCA-B, Oct-1 and Oct-2, yeast A/α, cErb-A, myc, mad and max, p53, mdm2, and others as set forth in Latchman, 1998, EUKARYOTIC TRANSCRIPTION FACTORS, 3^{rd} Ed., Academic Press: New York. Also provided by the invention are fusion protein derivatives of these or other regulatory proteins, wherein at least the DNA binding motif of the protein, which provides binding specificity, is fused to a small molecule regulator binding site, most preferably resulting in a fusion protein wherein binding of the small molecule regulator effects a structural change in the fusion protein that inhibits DNA binding to repressor regulatory proteins and enhances DNA binding to activator regulatory proteins. In still further embodiments of both the native and fusion protein derivatives of the regulatory proteins of the invention are provided embodiments thereof comprising a mammalian nuclear localization signal, such as the 7-amino acid nuclear localization signal of the SV40 T antigen (Kalderon et al.; 1984, Cell 39: 499-509). The regulatory protein provided by the invention can be endogenous to a cell comprising a recombinant promoter or recombinant expression construct of the invention, or can be an exogenously-introduced, heterologous regulatory protein, most preferably provided as a second recombinant expression construct that can be expressed, most preferably constitutively expressed, in said cell. Embodiments wherein a single recombinant expression construct comprising a gene operably linked to a genetically-engineered promoter element of the invention and a nucleic acid encoding a cognate regulatory protein are also encompassed by the invention.

As used herein the term “operator elements” is intended to encompass classic bacterial genetic “operators” as known in the art (see, for example, Stent & Calendar, 1978, MOLECULAR GENETICS, 2^{nd} ed., W. H. Freeman & Co.: San Francisco, Calif.), as well as mammalian transcription factor binding sites. Examples of such elements include but are not limited to the lac operator from the E. coli lactose operon (Muller-Hill, op. cit.), the CAP binding site, lambda phage early and late operator sequences, mammalian heat shock elements, cAMP responsive element, steroid-inducible elements, immunoglobulin octamer sequences, heavy metal responsive elements, serum responsive element binding site, MyoD binding site, homeobox, AP1, and any DNA sequence or motif that binds to a native or engineered regulatory protein. As provided herein, operator elements can be positioned in the genetically-engineered promoter elements of the invention at any site that effects functional transcription regulation mediated by the cognate regulatory protein, for example, in functional proximity to promoter features such as the TATA box, the CAAT box, enhancer elements, the transcriptional start site, SP1 sites, or any other functional topographic promoter element. In preferred embodiments, the constructs comprise enhancer elements that increase transcription, more preferably wherein the enhancer elements are tissue specific enhancer elements. As used herein, a “tissue-specific” enhancer element is an element that increases expression from a promoter only in cells of a tissue from which the enhancer is derived or in which the enhancer is specifically active. In additional embodiments, the constructs further comprise an intron, most preferably wherein the intron comprises a DNA fragment inserted into the construct between the operators and the start site of translation.

As used herein the term “operably linked” is intended to describe covalent linkage between nucleic acids wherein the quality, position and proximity of the linkage ensures coupled replication and is sufficient and, appropriate to be recognized by regulatory proteins and other trans-acting transcription factors and other cellular factors whereby polypeptide-encoding nucleic acid is efficiently expressed under appropriate conditions.

As used herein, the term “negative or reverse orientation to the direction of transcription” is intended to encompass an arrangement of the DNA operator sequences in the promoter construct whereby the art-recognized sequence of the operator (set forth in the 5′->3′ direction) is opposite to and in the reverse orientation to the direction of transcription from the plasmid (also set forth in the 5′->3′direction). Examples of said arrangement include, inter alia, the promoter construct denoted Op12 herein.

As used herein the term “trans-acting transcription factors” is intended to encompass polypeptides that, either themselves or as part of a multiprotein complex, recognize their cognate cis-acting transcription control elements and thereby mediate expression, particularly “inducible” expression as defined herein, of polypeptide-encoding nucleic acids operatively linked thereto. In preferred embodiments, the trans-acting transcription factors encoded by the recombinant expression constructs of the invention are derived from naturally-occurring regulons and thereby permit expression of recombinant polypeptides to be induced by altering cell culture conditions, for example, by adding an effective amount of the inducing agent into the culture media.

As used herein the term “promoter” is intended to encompass any nucleic acid that mediates expression of a gene to which it is operably linked in a cell, most preferably a mammalian cell. Expression via a promoter of the invention is typically by transcription of the gene sequence from an initiation site adjacent to the promoter, most preferably a site positioned between the promoter sequence and the protein-coding gene sequence. Representative and exemplary promoters comprise sequences such as AT-rich sequences termed “TATA” boxes, and additional sequences comprising the sequence “CAAT” that are recognized as mediating the interaction of the nucleic acid of the promoter with protein factors such as RNA polymerase. Non-limiting examples of promoters useful in the practice of the invention include bacterial-promoters such as the bacterial lactose operon promoter, viral promoters, including lambda bacteriophage promoters and particularly mammalian virus promoters, including promoters from SV40, adenovirus, adeno-associated virus, herpes simplex virus, cytomegalovirus, and retroviruses, and mammalian promoters, such as the immunoglobulin promoter, metallothionein promoter, thymidine kinase promoter, heat shock protein (hsp70, hsp83, hsp27) promoter, insulin promoter, growth hormone promoter, and albumin promoter.

As used herein, the term “tissue-specific promoter” is intended to encompass promoters that express tissue-specific genes, which are genes that are not expressed generally in all cells but are expressed predominantly in certain cell types. Non-limiting examples of tissue-specific promoters include but are not limited to the albumin and tyrosine hydroxylase promoters in liver cells, myosin light chain promoter in muscle cells, and globin promoters in reticulocytes.

The term “regulatable promoter” is intended to encompass DNA sequences that mediate transcription of a nucleic acid in a cell. In addition to the features and properties possessed by promoters generally, regulatable promoters are distinguished from promoters that are not regulatable in that regulatable promoters are operatively linked to “cis-acting transcription control elements” that will be understood to be nucleic acid sequences that regulate or control transcription of a polypeptide-encoding nucleic acid. As used herein, the term “cis-acting transcription control element” is particularly directed to nucleic acid sequences that make said regulatable promoter “inducible,” as that term is defined herein below. Said regulatable promoters of the invention comprising said cis-acting transcription control elements are operatively-linked to polypeptide-encoding nucleic acids and control transcription thereof in a cell, most preferably a yeast cell, into which a recombinant expression construct of the invention has been introduced. Most preferably the transcription control of the regulatable promoters of the invention is mediated by interaction between the cis-acting transcription control elements with the trans-acting transcription factors encoded by the recombinant expression constructs of the invention.

As used herein, the term “synthetic promoter” is intended to encompass promoter elements that are produced by chemical or in vitro biological synthesis and are useful in constructing promoters that are not found in a native state in a cell. In other usages, synthetic promoters are tissue-specific promoter elements that are engineered to resemble promoter elements from tissue specific genes from a particular tissue, and to provide tissue specificity for expression without affecting native, endogenous promoter elements or the regulation thereof.

The term “inducible” will be understood to mean that activation of transcriptional activity of a regulatable promoter comprising a cis-acting transcriptional control element is initiated or increased by a stimulus. Preferably, the inducing stimulus is an alteration in cell culture conditions, including but not limited to a change in temperature, density or the presence of a small molecule such as a metabolite, nutrient, or other small molecule regulator to the culture media.

As used herein the term “small molecule regulator” is intended to encompass any biologically-active molecule having a molecular weight of less than 1 kD that binds to and effects the function of a regulatory protein of the invention. Preferably, the small molecule regulator is a molecule that is not produced by the cell, tissue or animal in which cells comprising the recombinant expression constructs of the invention reside, nor is the molecule preferably a nutrient or metabolite thereof. Most preferably the regulator is a molecule foreign but not toxic to the cell, tissue or animal, and most preferably the small molecule regulator effectively enters cells or tissues containing the recombinant expression constructs of the invention. Non-limiting examples include gratuitous beta-galactosidase homologs such as X-gal (5-bromo-4-chloro-3-indolyl-β-D-galactopyranoside).

The recombinant expression constructs of the invention are useful for providing regulated, either inducible or repressible, expression of genes, preferably mammalian genes and most preferably genes for which modulation of expression provides a benefit, either in vitro (such as in maximizing the production of a recombinant protein) or in vivo (such as in enabling metabolic responsiveness of gene expression, for example, for constructs encoding insulin that are most preferably responsive to blood sugar levels). Such genes include but are not limited to insulin, growth hormone, cytokines, blood clotting Factor VIII, blood clotting factor XI, von Willebrand's factor, erythropoietin, thrombopoietin, tissue plasminogen activator, dystrophin, CFTR, Pgp, leptin, and proopiomelanocotrin.

The recombinant expression constructs of the invention are also advantageously provided wherein a reporter gene is operably linked to the genetically-engineered promoter of the invention. Suitable reporter genes include but are not limited to luciferase, beta-galactosidase, dihydrofolate reductase, thymidine kinase, chloramphenicol acetyl transferase, green fluorescent protein, hygromycin resistance, P-glycoprotein, neomycin resistance or any other gene whose expression provides a suitable means for phenotypic selection. Reporter-gene encoding recombinant expression constructs are useful, inter alia, for optimizing expression regulation by small molecule regulators.

The invention also provides recombinant cells comprising the recombinant expression constructs of the invention wherein regulated expression of a gene encoded by the construct can be achieved. In certain preferred embodiments, the cells are cell lines, either established cell lines such as HEK293 cells as are available, for example, from the American Type Culture Collection (Manassas, Va.) or primary cells and cell lines, such as primary cultures of fibroblasts, hematopoietic cells, and germ cells. In these embodiments, the recombinant expression constructs of the invention are introduced into the cells using methods well-known in the art, including but not limited to electroporation, transfection using calcium phosphate co-precipitate or lipid-mediated transfection, or viral infection. The choice of the method used to introduce the recombinant expression construct of the invention into a particular cell or cell line is within the skill of the ordinarily skilled worker and can be adapted to the cell or cell line without changing the character or effectiveness of the invention. In certain other embodiments, the cells comprise a tissue, either in vivo or ex vivo, and the recombinant expression constructs can be introduced into cells in the tissue either specifically (for example, by targeting certain cell types for infection or by targeting with lipids or liposomes with or without cell type-specific molecules embedded therein), or non-specifically, most directly by simple injection as disclosed in U.S. Pat. No. 5,580,859 (incorporated by reference herein). Alternatively, infection using recombinant adenovirus (as disclosed, for example, in U.S. Pat. No. 5,880,102, incorporated by reference herein), recombinant adeno-associated virus (as disclosed, for example, in U.S. Pat. No. 5,622,856, incorporated by reference herein), or recombinant retroviral vectors (as disclosed, for example, in U.S. Pat. No. 5,952,225, incorporated by reference herein) can be used. Alternative methods include electroporation (as disclosed, for example, in U.S. Pat. No. 5,983,131, incorporated by reference herein) and lipid or liposome-mediated introduction of exogenous DNA (as disclosed, for example, in U.S. Pat. No. 5,703,055, incorporated by reference herein). In certain embodiments of the recombinant cells of the invention the regulatory protein that recognizes and mediates expression regulation of genes encoded by the construct and operably linked to a genetically-engineered promoter of the invention is a protein endogenously produced by the cell, so that it is not necessary to introduce additional exogenous DNA into the cell. These embodiments are advantageous because only one exogenous construct must be introduced into the cell, and the endogenously-produced regulatory protein is in its proper cellular milieu. In alternative embodiments, such additional exogenous DNA encoding a regulatory protein that recognizes and mediates expression regulation of genes encoded by the construct and operably linked to a genetically-engineered promoter of the invention is introduced into the cell. In preferred embodiments this regulatory protein-encoding construct is an additional and separate construct from the recombinant expression construct of the invention encoding a gene of interest wherein expression thereof is regulated. In these embodiments, it is preferable to establish a cell line in which the construct encoding the regulatory protein is stably expressed. Alternatively, the nucleic acid encoding the regulatory protein is provided as part of the recombinant expression construct encoding the gene to be expressed in a regulated manner. These embodiments are advantageous because the complete regulatory cassette can be introduced into any cell or cell line, and the regulatory protein an be modified for nuclear localization, expression level or specificity for the gene expression-regulating genetic elements in the recombinant promoter.

The invention also provides methods for producing the genetically-engineered promoter elements of the invention. These methods utilize promoters that are otherwise not regulated, at least by the regulatory proteins cognate to the promoter elements provided by the invention. The methods of the invention preferably include identifying one or a plurality of conveniently-located, preferably unique restriction enzyme recognition sites, either naturally-occurring or genetically-engineered, such as by linker-scanning (as disclosed, inter alia, in Gustin et al., 1993, Virology 193: 653-660; Brown et al., 1992, Mol. Cell Biol. 12: 2644-2652; McKnight et al., 1982, Science 232: 316, incorporated by reference herein) or in vitro amplification techniques (as disclosed, for example, in Chumakov et al., 1991, Proc. Natl. Acad. Sci. USA 88: 199-203). Construction of the promoters of the invention is accomplished using standard ligation techniques performed under conditions of excess copies (relative to the copies of the promoter to be altered) of the promoter elements, such as operator elements, of the invention, provided as double-stranded oligonucleotides. Most preferably, the double-stranded oligonucleotides encoding the promoter elements of the invention are provided having a complementary restriction enzyme recognition site at each end, wherein the ends can be the same or different but most preferably “match” the restriction enzyme recognition sequences present at either end of the digested promoter. It will be understood that “complementary” restriction digested elements and promoters will ligate in a “sticky-end” manner, and that the resulting ligation product will either regenerate the restriction site or not (e.g., ligation of a BamHI/BglII combination). As performed herein, the resulting ligation mixture was found to contain reconstituted promoter elements having one or a plurality of regulatable control elements inserted in at least one position in the promoter corresponding to the site of restriction enzyme digestion.

The invention thus advantageously provides methods for producing the regulatable promoter elements without prior determination of the number of DNA operator sequences introduced into said promoter. As disclosed herein, this technique permits production of a plurality of different promoter constructs having an essentially random pattern of the number and orientation of the DNA operator sequences introduced into the promoter at positions defined by restriction enzyme digestion sites. These constructs can then be tested to determine, without prior knowledge or design, the constructs having beneficial or superior properties, including for example baseline expression levels, extent of inducibility, maximum inducible transcript levels, and other properties known to those with skill in the art. The method is thus a particularly advantageous aspect of the invention as disclosed herein.

The invention also provides methods for using the recombinant expression constructs and regulatory proteins cognate to the regulatable promoter sequences to produce a protein encoded by a gene operably linked to the regulatable promoter of the invention. In preferred embodiments, the method as provided is an in vitro method, wherein recombinant mammalian cells are used to produce desired proteins. These embodiments are advantageous, inter alia, because the resulting proteins are more properly post-translationally processed (e.g., by glycosylation or proteolytic cleavage) to produce a protein more similar to the native protein than typically recovered when using prokaryotic or yeast cells for producing the protein. In addition, the regulatable promoter permits the time for maximum expression to be selected by contacting the cell culture with a small molecule regulator. This allows the culture conditions (such as cell density) to be selected to optimize production conditions.

The methods of the invention also are applicable to cells and tissues ex vivo and in vivo. In these embodiments, the recombinant expression construct is introduced into a tissue, such as skeletal muscle, that is available and easily accessed. After introduction, it is expected that certain embodiments of the recombinant expression constructs of the invention will have a detectable level of basal expression of the protein encoded therein, whereas others will not. Co-introduction of a recombinant expression construct encoding the cognate regulatory protein, preferably to be expressed constitutively in the tissue, permits regulation of expression of the gene of interest by administering to a tissue ex vivo or an animal in vivo a small molecule regulator. These methods are particularly useful for providing regulated expression of genes such as insulin that respond and need to respond to metabolic stimuli.

In vivo applications of the methods of the invention require adjustment of the amount of the recombinant expression construct encoding the gene of interest that is administered and optimization of regulation of gene expression using small molecule regulators. Such optimization protocols are within the skill of those with skill in the medical arts.

The following Examples illustrate certain aspects of the above-described method and advantageous results. The following examples are shown by way of illustration and not by way of limitation.

### EXAMPLE 1

### Production of Recombinant HEK293 Cells Stably Expressing Bacterial lac Repressor Protein

In order to test recombinant promoter constructs of the invention for inducible expression, a cell line expressing the bacterial lactose repressor protein was necessary. Human embryonic kidney cells (HEK293 cells; A.T.C.C. Accession No. CRL-1573, American Type Culture Collection, Manassas, Va.) expressing the bacterial lactose repressor protein were produced as follows

The lacI gene encoding the lac repressor protein (LacR) was first amplified by PCR using pGEX-2T (Amersham Technologies, Piscataway, N.J.) as a template and then digested by XhoI/BamHI to produce a 1.109 kb fragment. This fragment was subcloned into the multiple cloning site of the CMV promoter-driven expression plasmid pcDNA3.1(−) (Invitrogen, Carlsbad, Calif.) after XhoI/BamHI digestion of the expression plasmid and ligation under conditions (excess expression plasmid) that favored production of the recombinant (see, Sambrook et al., 2000, MOLECULAR CLONING, 3^{rd} ed., Cold Spring Harbor Laboratory Press: N.Y.). The translation initiation codon in the lacI gene (GTG) was changed to an initiator methionine codon (ATG) and a short sequence encoding a nuclear localization signal from SV40 (CCAAAAAAGAAGAGAAAGGTA (SEQ ID No. 1), which encodes PKKKRKV; SEQ ID No. 2) was fused in-frame with the lac repressor gene immediately following the ATG codon by routine PCR amplification using a program consisting of an initial denaturation step at 94° C. for 30 sec, followed by 26 cycles of 94° C. for 30 sec, 50° C. for 30 sec and 72° C. for 1 mM.

The LacR repressor construct was transfected into HEK293 cells as follows. A proliferating culture of HEK 293 cells in logarithmic growth phase was transfected with the construct by lipofection using Lipofectamine reagent (Life Technologies, Bethesda, Md.) according to manufacturer's instructions. The cells were subsequently transferred to selective media containing 0.8 mg/mL G418. Individual G418-resistant clones were obtained by limited dilution and propagated for further analysis. From these clones a single clone, termed 5-D4 was selected based on expression of intact Lac repressor protein as detected by immunoblot analysis using anti-LacR antibodies (Upstate Biotechnology, Lake Placid, N.Y.).

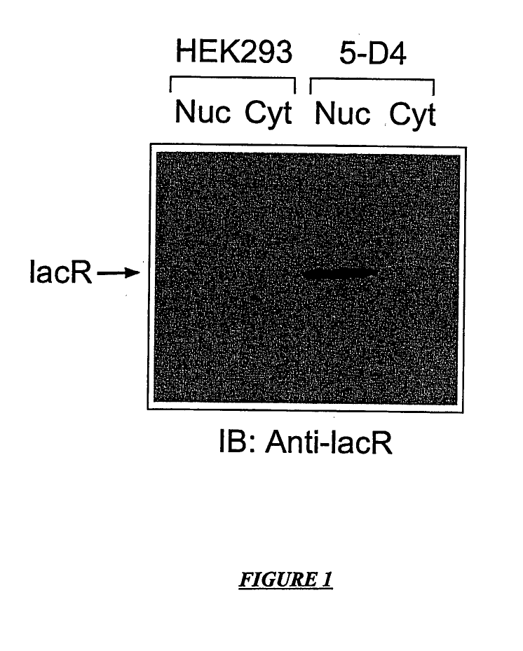

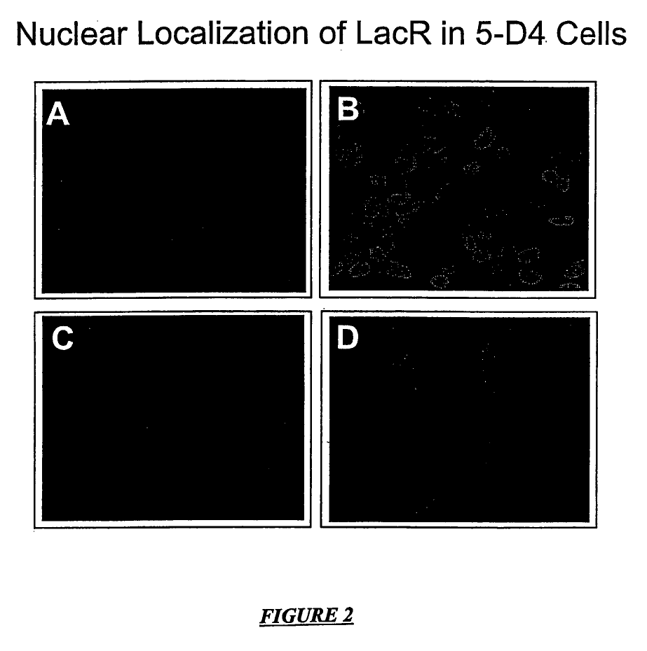

To verify that the LacR protein is expressed in 5-D4 cells and expressed in the cell nucleus, HEK-293 cells and 5-D4 cells were lysed and total cytoplasmic and nuclear fractions were individually separated by SDS-polyacrylamide gel electrophoresis (SDS-PAGE). Proteins were then transferred to a PVDF support membrane and probed with a mouse monoclonal anti-LacR IgG₁ antibody (clone 9A5; Upstate Biotechnology). The presence of immune complexes was detected by enhanced chemiluminescence using horseradish peroxidase-conjugated sheep anti-mouse IgG (Amersham, Piscataway, N.J.). Results of this analysis are shown in FIG. 1. Intact LacR protein is detected as a single, 40 kD band in the nuclear fraction but not the cytosolic fraction. Untransfected cells shown no LacR expression. These observations were further confirmed by immunostaining experiments; the results of which are shown in FIGS. 2A through 2D. In these experiments, anti-LacR antibody binding was detected by rhodamine red X-conjugated goat anti-mouse secondary antibody (Vector, Burlingame, Calif.). In the Figure, a distinctly nuclear pattern of immunostaining was detected in the nucleus of transfected cells (FIG. 2B), and is fully concordant with non-specific nuclear staining detected by 4′,6′-diamino-2-phenylindole (DAPI; FIG. 2D). In contrast, no immunostaining was observed when a combination of preimmune sera and rhodamine-conjugated secondary antibody was used (FIG. 2A), although cells are present as revealed by DAPI staining (FIG. 2C).

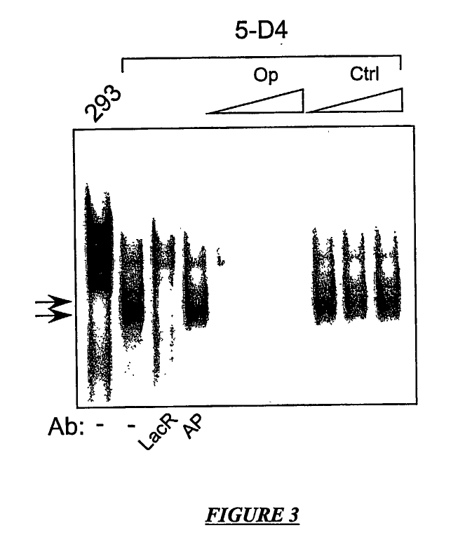

Electrophoretic mobility shift assays were performed to demonstrate that the LacR produced in 5-D4 cells were competent to bind lac operator sequences. Nuclear extracts from 5-D4 or untransfected HEK293 cells were incubated with ³²P-labeled double-stranded oligonucleotides having the sequence of the lac operator sequence and the resulting complexes separated by gel electrophoresis. The results of these experiments are shown in FIG. 3. At least two distinct protein-DNA complexes were detected in this assay (shown by arrows), and mobility shift of the ³²P-labeled double-stranded oligonucleotide can be completely inhibited by incubation with anti-:LacR antibodies. This inhibition is not observed if the extracts are incubated with irrelevant antibody (AP; lane 4). Formation of the complexes can also be competed with increasing concentration of unlabeled, double-stranded oligonucleotide having the lac operator sequence, but not oligonucleotides having an unrelated, control sequence.

These results showed that 5-D4 cells are a stably-transfected cell line that expressed high levels of immunoreactive LacR protein that is localized to the cell nucleus and capable of binding lac operator sequences.

### EXAMPLE 2

### Production of Op-SV40.LacZ Constructs

Recombinant SV40-derived promoter constructs operably linked to bacterial beta-galactosidase (LacZ) were produced as follows.

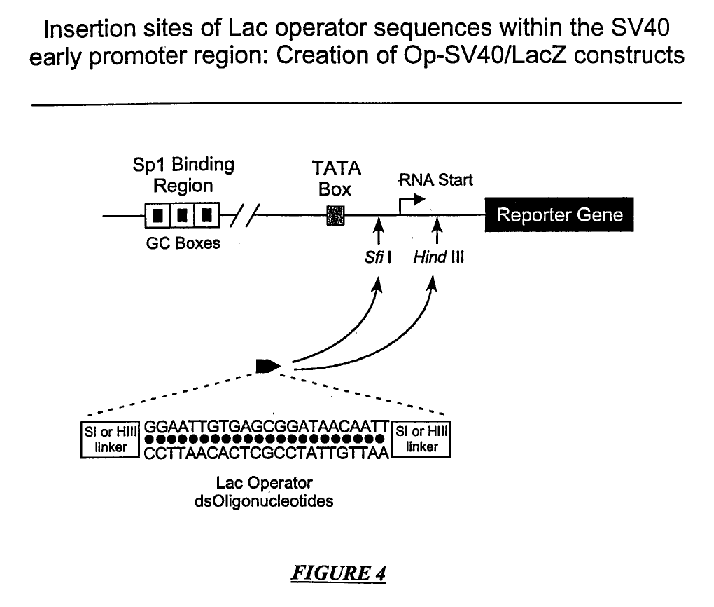

A plasmid containing the SV40 promoter operably linked to LacZ (termed pSVβ-gal; Promega, Madison, Wis.) was used to created the lac operator-containing recombinant expression constructs of the invention. A physical map of the promoter region of this plasmid is shown in FIG. 4. The transcription initiation site in the SV40 promoter in this plasmid is flanked by two unique restriction sites: SfiI (GGCCNNNNNGGCC) and HindIII (AAGCTT). The plasmid was digested with either or both of these enzymes, and then sticky-end ligated to—annealed, double-stranded, 5′ phosphorylated oligonucleotide(s) encoding the lac operator sequence having HindIII or SfiI linkers at each end of the oligonucleotide, or having HindIII linker at one end and an SfiI linker at the other end of the oligonucleotide. Ligation reactions were performed under conditions of molar excess of the annealed oligonucleotides, and the ligation reactions allowed to proceed at random to produce a plurality of ligation products having one or a multiplicity of copies of the lac operator oligonucleotide at either one or both restriction sites. The ligation reaction products were used to transform competent E. coli bacteria, plated, grown into individual colonies using LB plates containing 100 μg/mL ampicillin, and DNA prepared from small-scale liquid cultures produced by standard techniques.

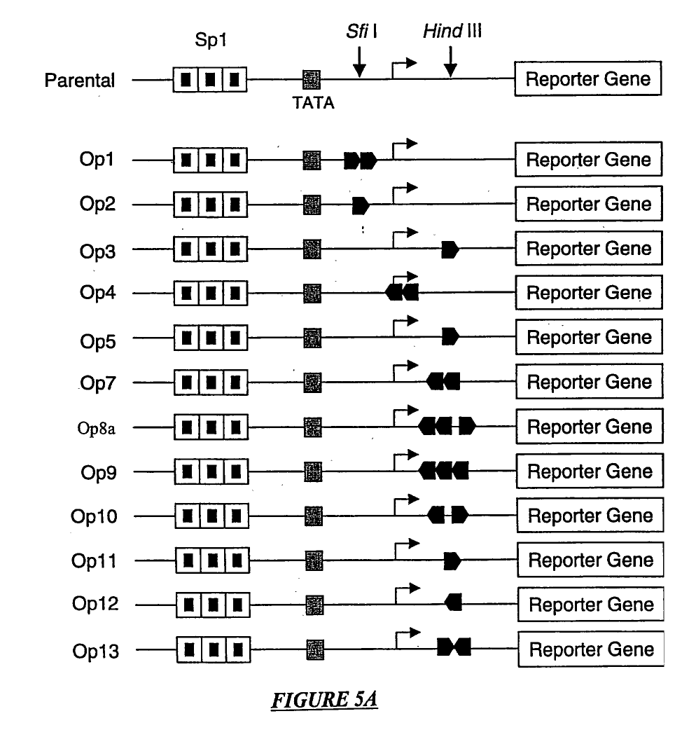

A number of different configurations of operator sequences comprising the SV40 promoter constructs were identified upon DNA sequence analysis of the resulting clones; some of these configurations are shown in FIGS. 5A and 5B (Op1 through 22). The majority of the ligation products comprised insertions into the HindIII site 3′ to the transcription initiation site in the promoter. Notably, one construct, Op4, contained two copies of the lac operator in negative orientation, ligating the two restriction sites and spanning the transcription start site. Op4 was also found to contain a deleted T (ΔT) in one of the two operator sequences inserted, as shown in FIGS. 5C and 5D.

Further screening for lac operator constructs produced another clone, Op8, that was found to demonstrate comparable inducible reporter expression as compared to Op4 (see Example 3 below). This clone contained two lac operators in the forward orientation inserted into the SfiI restriction enzyme site of pSV-β-gal using SfiI linkers (FIG. 5C). In this construct, one of the two lac operators contained a single nucleotide change (T->A). This plasmid was used to make additional constructs and assayed in expression experiments.

To further identify the effects of the deletion (ΔT) in the second lac operator of the Op4 construct, a plasmid was engineered that contained a single synthetic lac operator sequence in the reverse orientation, inserted into the SfiI-HindIII restriction enzyme sites with the same T deletion (ΔT) as in Op4 (FIG. 5C). This operator plasmid, Op4-6, was used in additional constructs and in expression experiments, and demonstrated comparable inducible regulation by IPTG to Op4 and Op8.

These constructs were tested to determine the capacity of LacR to regulate transcription from the SV40 promoter as described in Example 3.

### EXAMPLE 3

### Identification of IPTG-Inducible Op-SV40/LacZ Constructs

The constructs identified in Example 2 were tested to demonstrate whether they could be induced to express beta-galactosidase in 5-D4 and C2C12 cells.

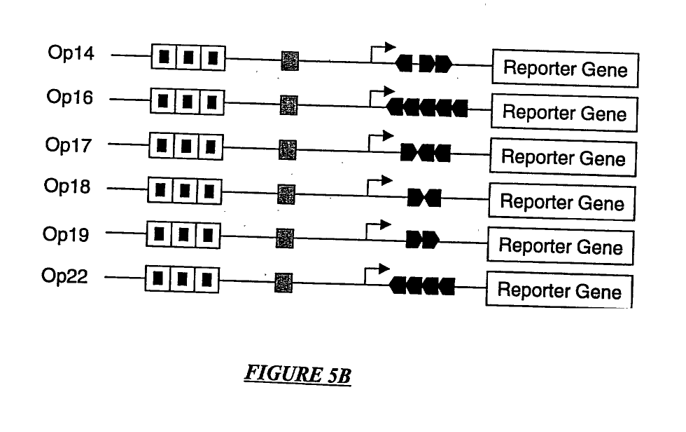

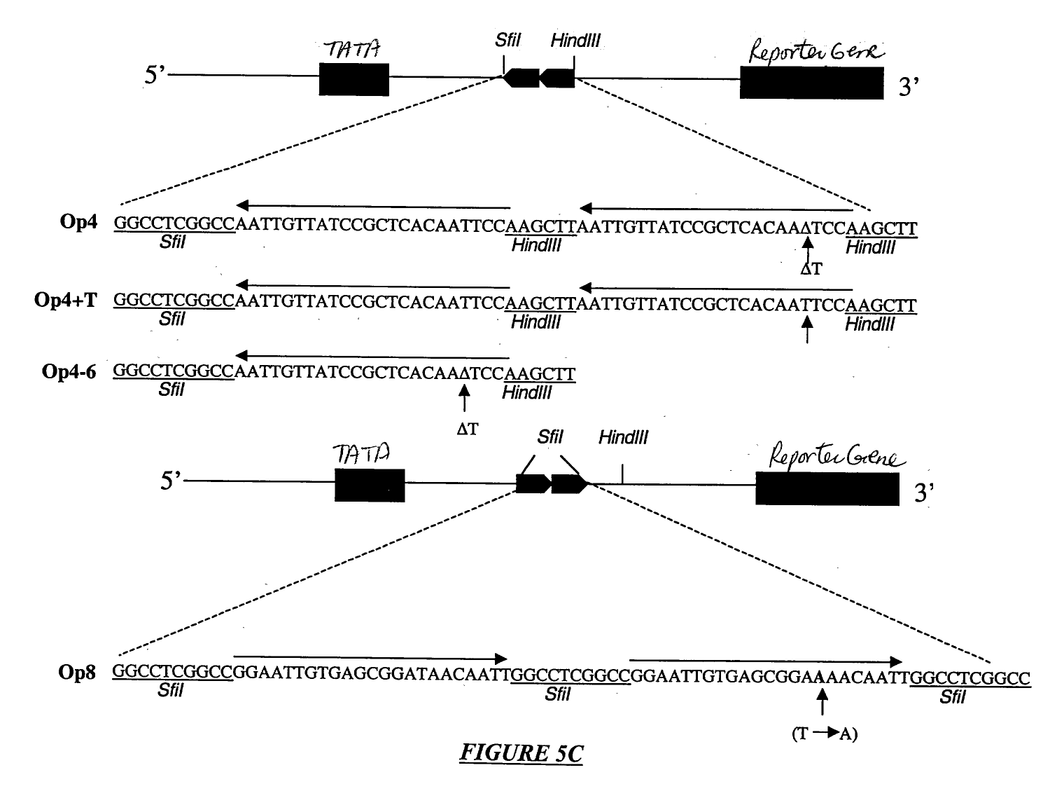

The Op-SV40/LacZ constructs shown in FIGS. 5A through 5C were transiently transfected into 5-D4 cells grown in 96-well plates (Costar), and 5 mM isopropylthiogalactoside (IPTG) was added to the cells 24 hours post-transfection. IPTG was not added to control cells. Beta-galactosidase (LacZ) expression was monitored after incubation for an additional 24 hours by staining with the chromogenic substrate X-gal (5-bromo-4-chloro-3-indolyl-β-D-galactopyranoside). The number of cells showing blue color expected from beta-galactosidase cleavage of X-gal were counted in each well and calculated as the mean value of duplicate wells. This data is shown in histogram form in FIG. 6A. The results shown in FIG. 6A demonstrated that each of the different constructs showed significantly different capacities to induce beta-galactosidase activity in the presence of IPTG (compared to the control, operatorless pSVP-gal parent plasmid).

One construct in particular, designated Op4, showed a reproducible pattern of IPTG-induced LacZ expression and was selected for further characterization. After transient transfection with Op4 and derivatives (described in more detail below) into 5-D4 cells grown in 6-well plates, total soluble cell extracts were prepared and the enzymatic activity of the LacZ contained therein was determined directly using a colorimetric method (Promega, Madison, Wis.). Briefly, cells were washed 2-3× in PBS without Mg⁺⁺ or Ca⁺⁺, then lysed in 1/10 volume (0.2 mL) 1× lysis buffer, and incubated for approximately 15 min. Lysates were then transferred to a microcentrifuge tube, on ice, and vortexed for 10-20 seconds. The cell lysates were spun at top speed for 2 min. in a 4° C. microcentrifuge and the cleared lysate was assayed either immediately or after storage at −70° C. 50 μL from each lysate was assayed according to the manufacturer's instructions (Promega's β-Galactosidase Enzyme Assay System with Reporter Lysis Buffer) in a standard 96-well plate by incubation with an equal amount of 2× assay buffer for 1-3 hours at 37° C., then compared to a β-galactosidase standard curve. Protein determination of the lysates allowed calculation of the amount of specific activity (μUnits β-galactosidase/mg total protein), normalized to background (mock-transfected HEK293) and β-galactosidase activity (pSV-β-gal control vector).

A detailed analysis of the structure and sequence of Op4 showed two copies of the lac operator arranged in tandem and in negative orientation (relative to the direction of transcription) between the HindIII and SfiI sites, as shown in FIG. 5C. In addition, a deletion of an A-T basepair was detected towards the 3′ end of the entire insert. To verify that the IPTG induction profile observed using the Op4 construct was entirely due to the inserted lac operator sequence, a similar construct was engineered in which a synthetic oligonucleotide based on the exact sequence of the Op4 insert was annealed and ligated into the HindIII and SfiI sites of pSVβ-gal; this construct was called Op4-1. The behavior of this construct, and particularly its IPTG induction profile, was indistinguishable from the original Op4 construct. Restoration of the deleted A-T basepair in construct Op4-T resulted in a markedly reduced efficiency in response to IPTG treatment. As shown in FIG. 6B. Op4 and certain derivatives were capable of significant, IPTG-inducible beta-galactosidase activity. Upon addition of IPTG, maximal expression of LacZ resulted in beta-galactosidase levels equivalent to the levels detected in the control, parent plasmid. To identify whether the deleted T or the change in transcription location affected expression and activity, a single copy of the lac operator with the identical T deletion was constructed as described above. The resulting plasmid construct is Op4-6.

To compare these IPTG-inducible constructs, Op4, Op4-6, and Op8 were transfected into either HEK-293 cells stably expressing the lacI repressor (5-D4) or C2C12 cells transiently co-transfected with the lacI repressor plasmid (LacR) in a 3:1 ratio of operator:repressor, as described above. At 24 h post-transfection, 5 mM IPTG was added to the transfected wells. Cells were washed and harvested at 48h, and total soluble cell extracts were prepared. Enzymatic activity of the reporter gene product, β-galactosidase, was determined by colorimetric methods, as described above.

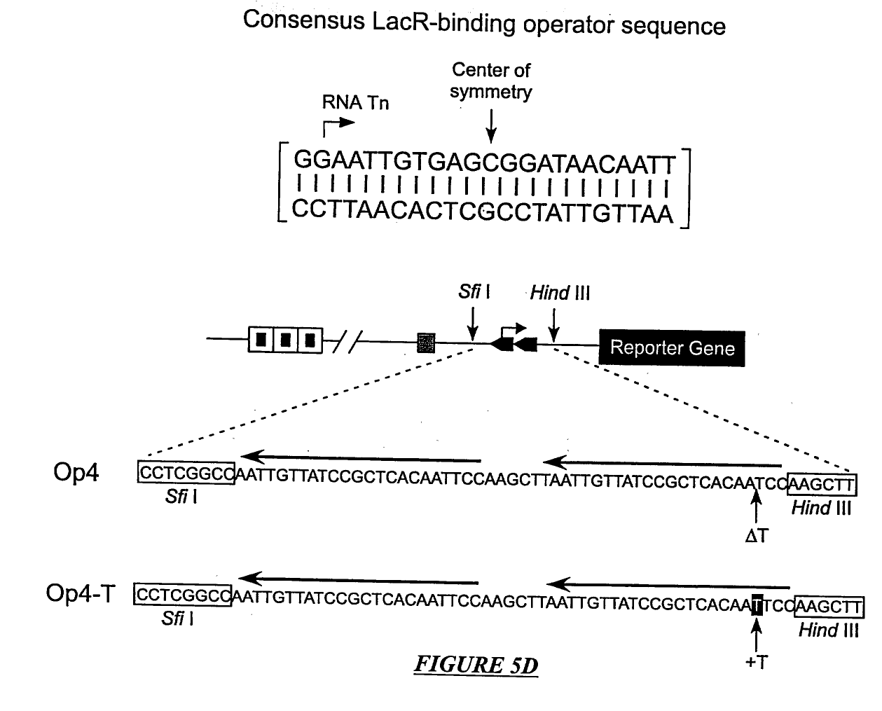

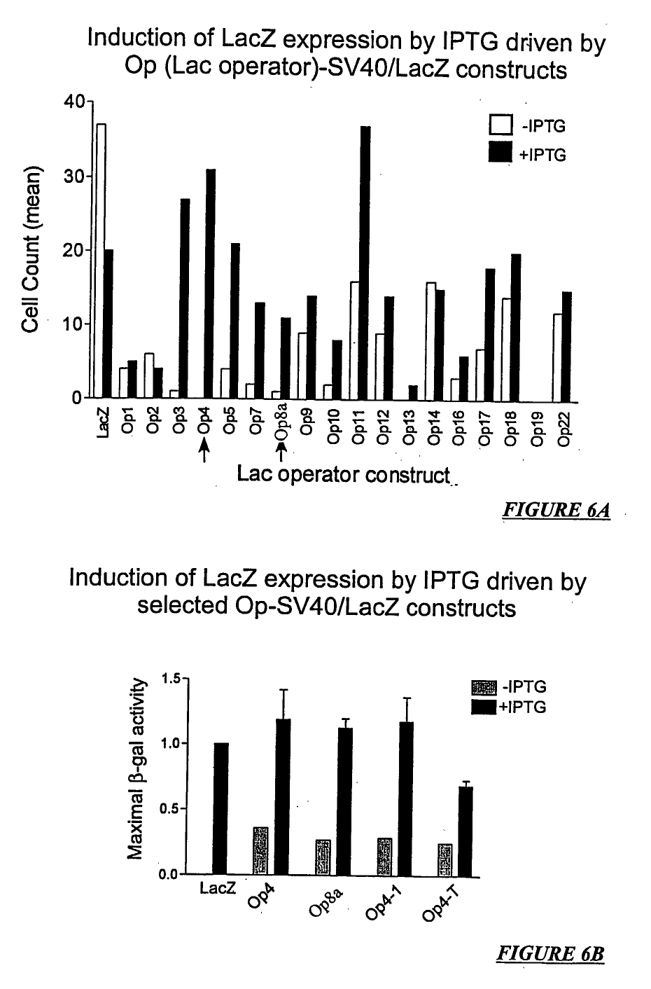

As shown in FIGS. 7A and 7B, all three constructs are capable of repression, presumably a result of binding to the lac repressor, and all achieve maximal expression upon addition of IPTG. This expression was compared to pSV-β-gal reporter in both the 5-D4 stable cell line and in C2C12 myoblasts. Background expression (mock-transfected HEK-293) was subtracted from expression in LacR-expressing cells (5-D4), and specific activity was normalized to levels of β-galactosidase.

These result demonstrated that specific configurations of lac operator sequences exist which cause efficient induction of gene expression from the promoter in cells expressing LacR and contacted with IPTG. These and other configurations can be produced according to the methods of the invention.

During construction of the lac operator constructs the transcriptional topography of the promoter was changed. For example, cloning of the lac operator sequences into Op4 ablated the pSV-β-galactosidase transcriptional start site. In order to characterize expression from the recombinant promoter, rapid amplification of 5′ cDNA ends (RACE) was performed to determine the new transcriptional start site of mRNA produced by the promoter using a commercially-available kit (GeneRacer, Invitrogen, Carlsbad, Calif.) used according to the manufacturer's instructions. Briefly, total RNA was isolated from HEK-293 cells transfected with Op4 DNA and harvested after 48 h. Cells (3×10⁷/plate) were washed three times with phosphate buffered saline (PBS) and RNA was isolated using TriReagent (obtained from Molecular Research Inc., Cincinnati, Ohio). Op4 RNA was dephosphorylated with calf intestinal phosphotase (CIP), then decapped using tobacco acid pyrophosphatase (TAP). The resulting RNA was ligated with T4 RNA ligase to a RNA linker and RT-PCR amplified using both the kit complementary oligomer and a gene-specific primer (pSV/R1:5′-GGCATCAGTCGGCTTGCGAGTTTACGTGCA; SEQ ID NO. 3). The cDNA was further amplified with a GeneRacer primer and a gene-specific primer (pSV/R1:5′-ATCTGCAACATGTCCCAGGTGACG; SEQ ID NO. 4), and the resulting product cloned into vector pCR4.0 (Invitrogen). Miniprep DNA was prepared and sequenced on several clones.

Sequence analysis of these clones determined that the transcriptional start site for Op4 begins at nt 337, an adenine (A), located between the TATA box and the two lac operators, upstream from the SfiI restriction enzyme site. This is also upstream of where the unaltered pSV-β-gal transcriptional start site occurs.

The transcriptional start site in the Op4 construct was further characterized by RNase protection assay (RPA) to confirm that the transcriptional start site in Op4 had been modified. A 305 bp fragment from Op4, containing the two lac operator sequences, was PCR amplified and cloned into the EcoRI/BamHI sites of plasmid Bluescript pSK+. (Stratagene, La Jolla, Calif.) From this construct (pSK+/Op4) a 501 bp PvuI/XhoI fragment, containing the 305 bp from Op4, was excised and radiolabeled with α-³²P-UTP (800 Ci/mmol) for use as a probe in the Maxiscript in vitro transcription assay (using a HybSpeed RPA kit obtained from Ambion, Austin, Tex.). RPA hybridization was performed using 1×10⁵ cpm of labeled probe (Op4) and 10 μg RNA (isolated as describe above for RACE assay). Following hybridization, RNase inactivation, and precipitation of protected RNA, pellets were resuspended in 8 μL gel loading buffer II and heated at 95° C. for 5 min. The samples were loaded onto a prerun (250V for 1 h) 4% acrylamide/8M urea gel, then electrophoresed for 1.5-2 h at 250V (0.75 mm) in 1×Tris-borate-EDTA buffer. The gel was vacuum-dried, then exposed to film at −80° C. overnight. Mouse β-actin RNA (5×10³ cpm) was used as an internal control.

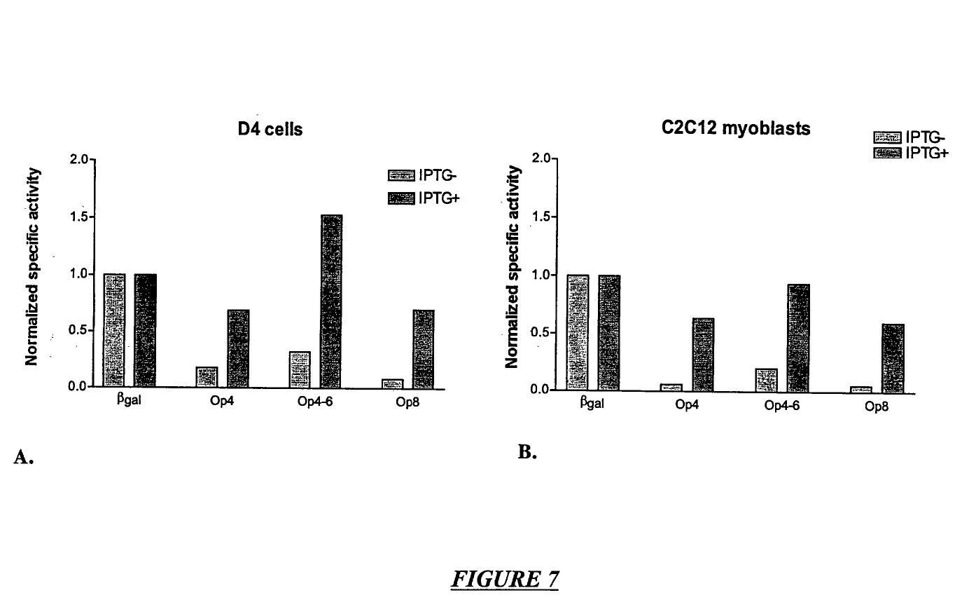

The 501 bp radiolabeled pSK+/Op4 RNA probe protected a fragment of 305 nt for Op4 and 243 nt of pSV-β-galactosidase (FIGS. 8A through 8C). This experiment substantiated that the start site of transcription was altered upstream of the operator sequences in Op4 as compared to pSV-β-galactosidase, as indicated in the Op4 5′ RACE analysis.

### EXAMPLE 4

### Vectors for Increasing Expression of lac Operator Constructs

Cis-acting myogenic factors in muscle cells are known to modulate the level of endogenous gene expression. When engineered into a reporter plasmid, these enhancer elements can increase the level of expression of the reporter gene in myotubes in vitro (Donoghue et al., 1988, Genes Dev. 2: 1779-90). To increase the level of expression of the reporter gene product β-galactosidase in the lac operator constructs of the invention, the muscle enhancer MLC1 and the SV40 intron were engineered into Op4, Op4-6, Op8.

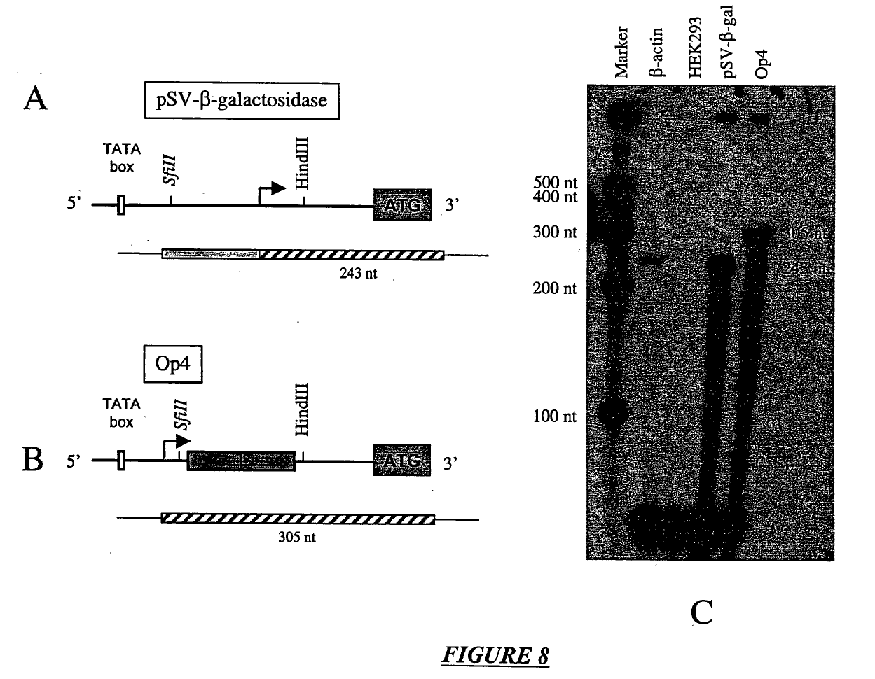

The 161 bp myosin light chain (MLC1) human muscle enhancer (Donoghue et al., 1988, Id.; SEQ ID NO. 5), containing myocyte-specific enhancer binding factors MEF-1 (Ebox) and MEF-2 (shown in FIG. 9), was inserted into the BamHI and PstI restriction enzyme sites in the 3′ UTR of Op4, Op4-6, and Op8 in the forward direction. This element was amplified from human genomic DNA using the two primers listed below. Forward primer:

| | |
|---|---|
| Forward primer: | SEQ ID NO.6) |

5′-CG(BamHI)CTAACCTTATTAAATTACCATGTG-3′

| | |
|---|---|
| Reverse primer: | (SEQ ID NO.7) |

5′-ACT(PstI)AAAAGTTATTTTTAAAGACTGAGGAATTAGG-3′

The 172 nt intron from plasmid pCMV-β (Clontech, Palo Alto, Calif.), containing the SV40 splice donor/acceptor (16s/19s) signals (Rosenthal et al., 1990, Nucleic Acids Res. 18: 6239-46), was inserted into Op8 and Op4 by digestion of pCMVβ-gal (Clontech, Palo Alto, Calif.) with XhoI/NotI restriction enzymes. The 180 bp fragment

5′-GAACTGAAAAACCAGAAAGTTAACTGGTAAGTTTAGTCTTTT
TGTCTTTTATTTCAGGTCCCGGATCCGGTGGTGGTGCAAATCAA
AGAACTGCTCCTCAGTGGATGTTGCCTTTACTTCTAGGCCTGTA
CGGAAGTGTTACTTCTGCTCTAAAAGCTGCGGAATTGTACCC-3′

(SEQ ID NO. 8) was gel purified and blunted with T4 polymerase. The lac operator constructs were digested with HindIII, filled in with T4 polymerase, dephosphorylated with calf alkaline phosphatase and ligated with the intron sequence. Clones were selected and sequenced to verify the correct direction 5′->3′ of the insert. This insert resulted in the intron inserted between the promoter and the reporter gene, proximal to the start of translation.

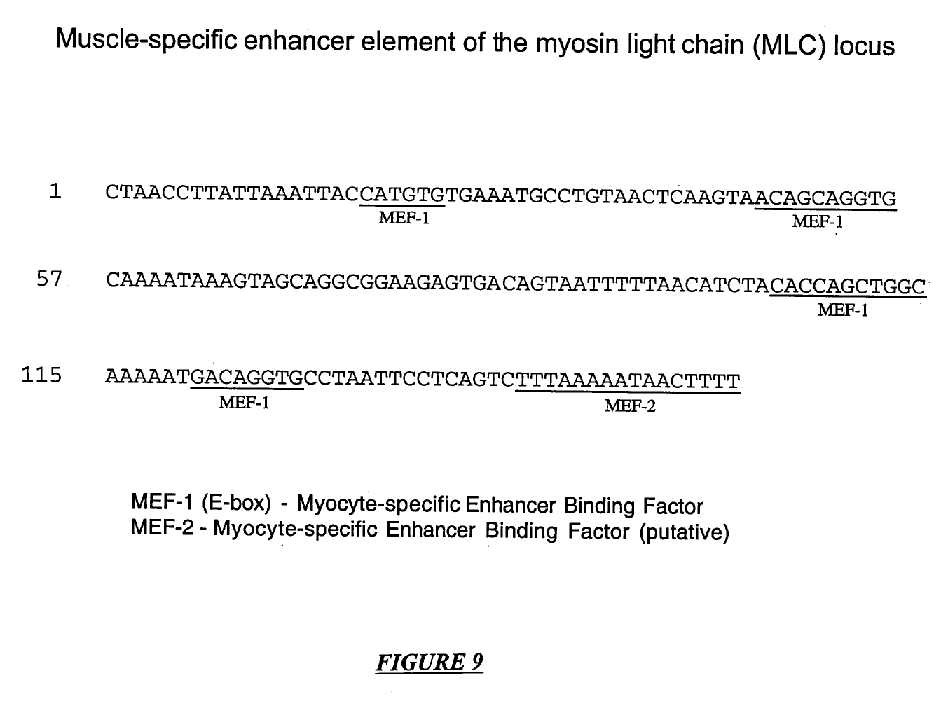

The three constructs containing the human muscle enhancer MLC1 (ME), the SV40 intron splice site (i), or both enhancer and intron (iME) were transiently co-transfected with the lacI repressor plasmid in a 3:1 ratio of operator:repressor, into C2C12 myoblasts, then differentiated for 14 days with 10% horse serum into myotubes. IPTG was added 24 h pre-harvest, and total soluble cell extracts were prepared as previously described. FIG. 10 shows repressed (−IPTG) and derepressed (+IPTG) β-galactosidase activity in both Op8 and in Op4-6, with a combination of elements, normalized to reporter gene expression alone. In all constructs, the addition of either the intron or the muscle enhancer increased the level of expression at least two-fold, and when both were engineered into the lac operator plasmid, the level of expression was higher than when only one element was present.

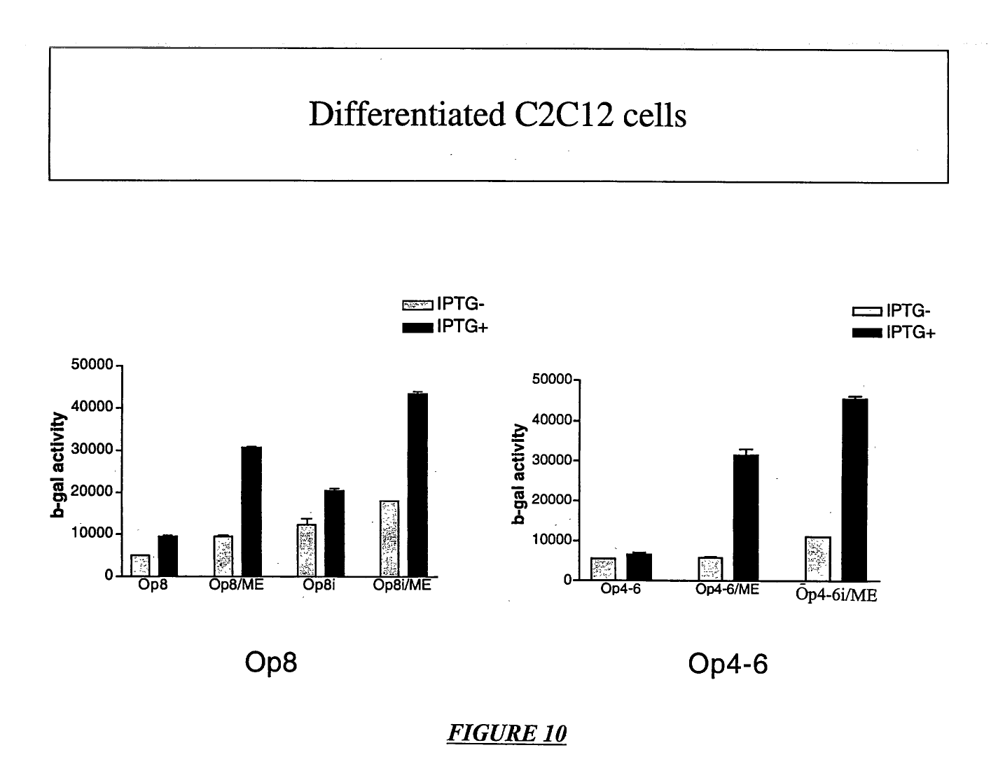

The time course of IPTG induction of β-galactosidase expression in one of the muscle enhancer and SV40 intron containing constructs was determined. The kinetics of Op8iME induction in C2C12 myoblasts were plotted over the course of 48 hours. Op8iME and the Lac-R were transiently cotransfected into C2C12 myoblasts. Sixteen hours post-transfection, 5 mM IPTG was added. β-galactosidase expression was monitored in cell lysates by chemiluminescent assay (Promega) at a selection of time-points from 1 h to 48 h. IPTG-dependent induction of β-galactosidase began at 4h and increased over time, with a maximum peak at 24 h (FIG. 11).

These results indicated that transfected cells responded to IPTG treatment with a rapid and sustained expression of the reporter gene while repression without IPTG remained low over time, and that optimal IPTG induction is between 24 and 48 hours.

### EXAMPLE 5

### Vectors Comprising Synthetic Promoter (SP) Constructs

Previous studies have shown that a synthetic chicken skeletal α-actin promoter caused increased gene expression in muscle cells in vitro and in vivo (Li et al., 1999, Nature Biotechnology 17: 241-245). Although this construct was based on the chicken skeletal α-actin promoter sequence, these promoters have a high level of conservation between species (Li et al., 1999, Id.). Accordingly, a synthetic human skeletal α-actin promoter (hskA) was used in combination with the recombinant expression constructs of the invention to optimize tissue-specific expression in the lac operator reporter constructs.

A 112 bp XhoI-BglII restriction fragment comprising a portion of the hskA promoter (from positions −88 to +24), containing the TATA site, was cloned into the pGL3 basic luciferase reporter plasmid (Promega). Clones were selected and analyzed for the correct sequence. Thereafter, 5′-phosphorylated oligonucleotides spanning both strands of the synthetic promoter (SP) were made (Invitrogen) having the sequence:

| | |
|---|---|
| 5′P CTAGCTCCGCCCTCGGCACCATTCCTCACGACACCCAAATATGGCGACG | (SEQ ID NO 8) |
| GGTGAGGAATGGTGGGGAGTTATTTTTAGAGCGGTGAGGAATGGT-3′ | |
| 5′P-GGCAGGCAGCAGGTGTTGGCGCTCTAAAAATAACTCCCGGGAGTTATT | (SEQ ID NO.9) |
| TTTAGAGCGGTGAGGAATGGTGGACACC-3′ | |
| 5′P-CAAATATGGCGACGGCACCATTCCTCACCCGTCGCCATATTTGGGTGTC | (SEQ ID NO.10) |
| CCGTCCGCCCTC-3′ | |
| 5′P-TCGAGAGGGCGGACGGGACACCCAAATATGG-3′ | (SEQ ID NO.11) |
| 5′P-CGACGGGTGAGGAATGGTGCCGTCGCCATATTTGGGTGTCCACCATTCC | (SEQ ID NO.12) |
| TCACCGCTCTAAAAATAAC-3′ | |

All primers were annealed at an equal molar ratio, then cloned into the NheI and XhoI restriction enzyme sites of pGL3 basic vector containing the partial hskA. The synthetic promoter contains the following sequence:

Sequence of SP/hskA Promoter (Upper Strand)

| | |
|---|---|
| (NheI)TCCGCCCTCGGCACCATTCCTCACGACACCCAAATATGGCGACGG | (SEQ ID NO.13) |
| GTGAGGAATGGTGGGGAGTTATTTTTAGAGCGGTGAGGAATGGTGGGC | |
| AGGCAGCAGGTGTTGGCGCTCTAAAAATAACTCCCGGGAGTTATTTTTA | |
| GAGCGGTGAGGAATGGTGGACACCCAAATATGGCGACGGCACCATTCC | |
| TCACCCGTCGCCATATTTGGGTGTCCCGTCCGCCCT(XhoI)aagggcagcgacatt | |
| cctgcggggtggcgcggagggaatcgcccgcgggctatataaaacctgagcagagggacaagcggccaccgcag | |
| cggacagcgccaagtgagatctgggg(BglII) | |

In this representation of the promoter (SP/hskA promoter), the synthetic promoter region is capitalized, and the partial hskA promoter is set forth in small letters. Restriction enzyme sites are shown in parentheses.

DNA was isolated and analyses confirmed the sequence for each construct used in expression experiments.

Synthetic Promoter Expression in Muscle Cells

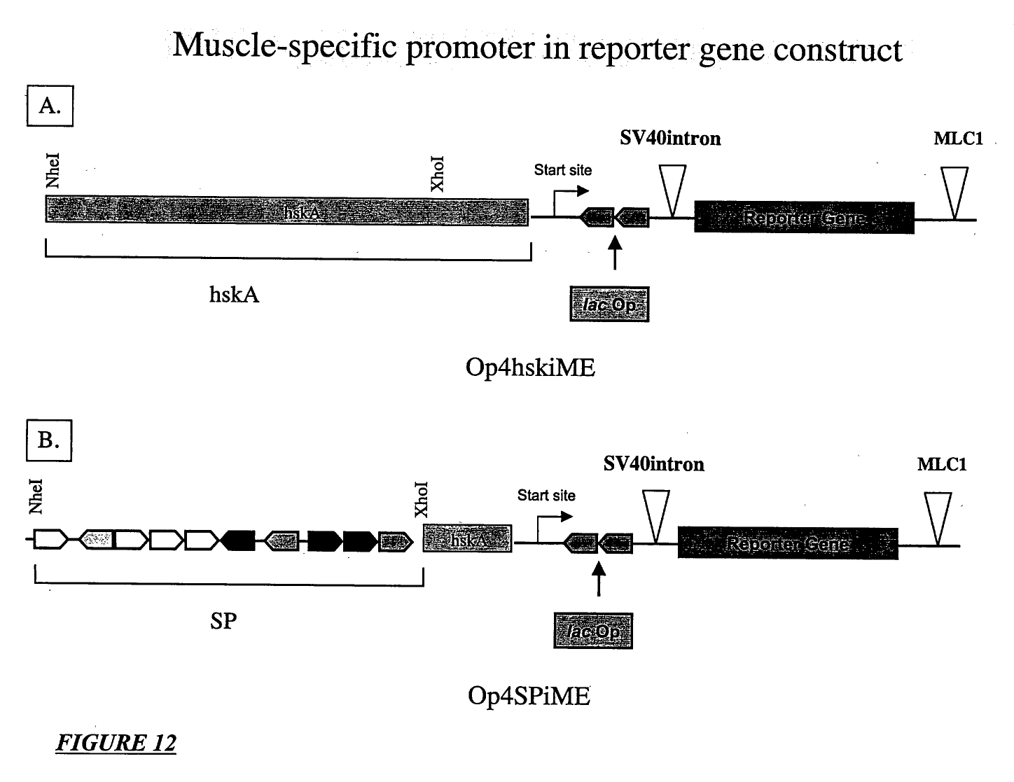

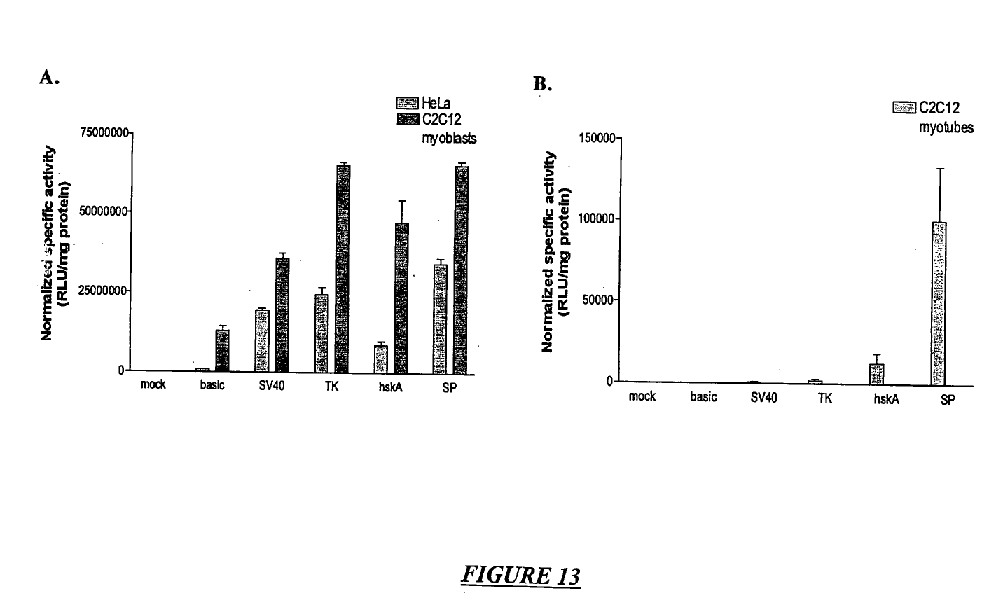

Expression of the reporter gene product in the luciferase reporter system was determined after transient transfection of the SP or control DNAs into HeLa cells and C2C12 myoblasts after 48 h (FIG. 13A) or in C2C12 myotubes after 14 d (FIG. 13B). IPTG was added 24 h pre-harvest, and total soluble protein obtained as previously described.

In myoblasts and in HeLa cells, all promoter-containing constructs transfected (SV40, thymidine kinase (TK), hskA, SP) showed higher specific activity than those cells transfected with either a promoterless plasmid (pGL3 basic) or mock-transfected. Expression was higher in tissue-specific myoblasts than in HeLa cells., In 14 d myotubes, the control plasmids containing the SV40 or the thymidine kinase (TK) promoter and enhancer expressed 7- to 10-fold higher than basal (mock or basic), while the tissue-specific hskA promoter plasmid expressed 10-fold higher than either the SV40 or the TK promoter. Notably, specific activity for the skeletal muscle-specific SP construct was 150-fold higher than that of the SV40 promoter.

The annealed chimeric/synthetic hskA promoter was then cloned into the NheI-BglII site of a promoterless Op4iME or Op8iME construct. Briefly, Op4iME and Op8iME were PCR amplified using primers having the sequence:

| | | |
|---|---|---|
| SPOpBglII/F: | 5′-(BglII) ATGCAGAGGCCGAGGCCGCCTCG-3′ | (SEQ ID NO.15) |
| SPOPNheI/R: | 5-(NheI) TGCTGCGCCGAATTCGTAATCATGTC-3′ | (SEQ ID NO.16) |

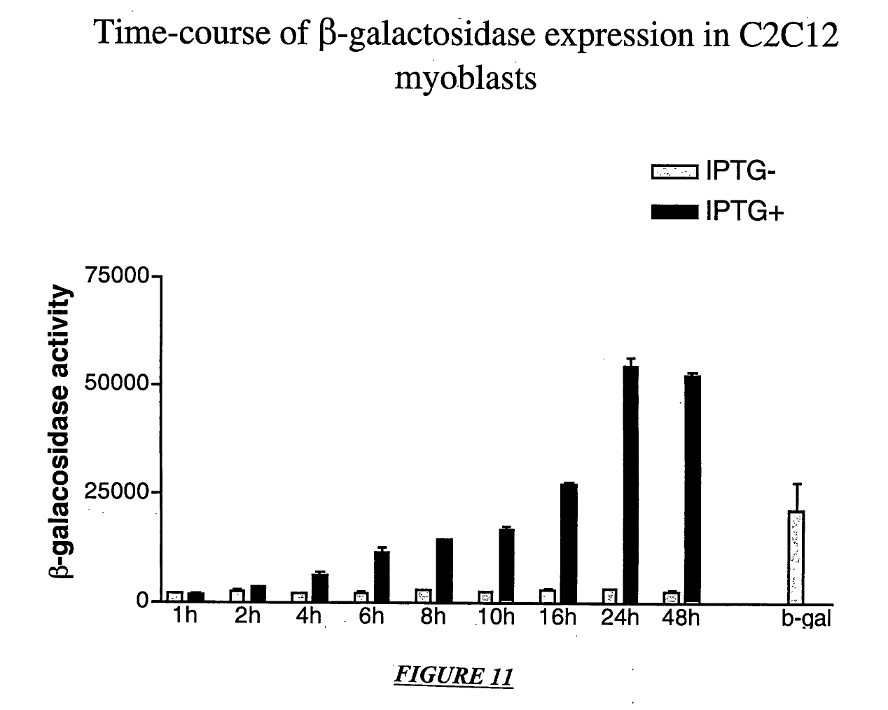

After restriction digest with these two enzymes, dephosphorylation, and purification of the amplified product, the annealed SP/hskA was cloned into this site. These new constructs were then transformed into E. coli strain DH5α for amplification of plasmid DNA. A schematic diagram of the Op4hskiME and Op4SPiME promoters are shown in FIGS. 12A and 12B.

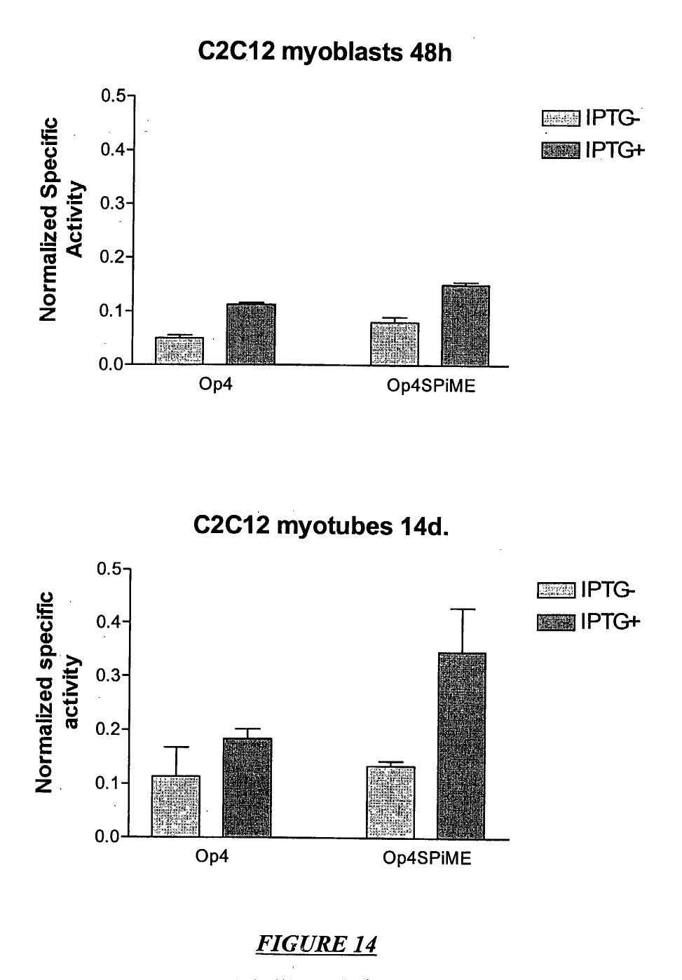

Results of Op8SPiME (not shown) and Op4SPiME assayed in transiently transfected C2C12 cells showed that after normalization to β-galactosidase and mock-transfected cells, the SP is active in the inducible lac operator/repressor system, and is specific for human skeletal muscle tissue (FIGS. 14A and 14B). Expression of the reporter gene is significantly higher in cells differentiated in vitro (myotubes) after approximately 14 days

These results demonstrated that the synthetic promoter recombinant expression constructs of the invention could be used for inducible expression of reporter genes in a variety of human cell types in vitro.

It should be understood that the foregoing disclosure emphasizes certain specific embodiments of the invention and that all modifications or alternatives equivalent thereto are within the spirit and scope of the invention as set forth in the appended claims.

We claim:

1. A recombinant expression construct encoding a gene operably linked to a promoter comprising transcriptional control elements that are responsive to a small molecule regulator, wherein expression of said gene is regulated by contacting a cell containing the recombinant expression construct with the small molecule regulator, and wherein regulated expression is mediated by at least two DNA operator sequences inserted upstream of the start of translation and downstream from the promoter TATA site and the start of transcription.

2. A recombinant expression construct according to claim 1, wherein the gene is a reporter gene.

3. A recombinant expression construct according to claim 2 wherein the reporter gene is luciferase, beta-galactosidase, dihydrofolate reductase, thymidine kinase, chloramphenicol acetyl transferase, green fluorescent protein, hygromycin resistance, P-glycoprotein, or neomycin resistance.

4. A recombinant expression construct according to claim 1, wherein the gene is a mammalian gene.

5. A recombinant expression construct according to claim 4, wherein the gene encodes insulin, growth hormone, a cytokine, blood clotting Factor VIII, blood clotting factor XI, von Willebrand's factor, erythropoietin, thrombopoietin, tissue plasminogen activator, dystrophin, CFTR, Pgp, leptin, or proopiomelanocotrin.

6. A recombinant expression construct according to claim 1, wherein the gene encodes an antigen.

7. A recombinant expression construct according to claim 6, wherein the antigen is a tumor antigen, a viral antigen, a bacterial antigen or a protozoal antigen.

8. A recombinant mammalian cell comprising the recombinant expression construct of claim 1, 2, 3, 4, 5, 6 or 7.

9. A mammalian cell according to claim 8, wherein the mammalian cell is a skeletal muscle cell.

10. A skeletal muscle cell according to claim 9, wherein the cell further comprises a tissue.

11. A recombinant mammalian cell according to claim 8 further comprising a transcriptional regulatory protein that mediates transcriptional regulation of the promoter by recognizing said operator sequences.

12. A recombinant mammalian cell according to claim 11, wherein the transcriptional regulatory protein is encoded by a DNA heterologous to said cell.

13. A recombinant mammalian cell according to claim 12, wherein the heterologous DNA encodes a bacterial transcriptional regulatory protein.

14. A recombinant mammalian cell according to claim 13, wherein the bacterial transcriptional regulator protein is the lac repressor.

15. A recombinant mammalian cell according to claim 14, wherein the lac repressor further comprises a mammalian nuclear localizing sequence.

16. The recombinant mammalian cell of claim 15, wherein the mammalian nuclear localizing sequence is the nuclear localizing sequence of SV40 T antigen.

17. A recombinant mammalian cell according to claim 12, wherein the heterologous DNA encodes a eukaryotic transcriptional regulatory protein.

18. A recombinant mammalian cell according to claim 12, wherein the heterologous DNA encodes a mammalian transcriptional regulatory protein.

19. The recombinant mammalian cell of claim 18 wherein the mammalian transcriptional regulatory protein comprises at least a DNA binding domain and a regulatory, ligand-binding domain.

20. A method for producing regulated expression of a gene in a mammalian cell, the method comprising the steps of introducing the recombinant expression construct of claim 1 into a cell further comprising a transcriptional regulatory protein that mediates transcriptional regulation of the promoter by recognizing said operator sequences and contacting the cell with an effective amount of the small molecule regulator.

21. A method for producing regulated expression of a gene in an animal, the method comprising the steps of introducing the recombinant expression construct of claim 1 and a second construct encoding a transcriptional regulatory protein that mediates transcriptional regulation of the promoter by recognizing said operator sequences into the animal and administering to the animal an effective amount of the small molecule regulator.

22. A method for producing a recombinant promoter element under transcriptional control of a transcriptional regulator protein, comprising the step of digesting DNA encoding a promoter with a restriction enzyme having a recognition site proximal to an mRNA transcription initiation site, mixing the digested DNA with an excess of a double-stranded oligonucleotide encoding transcription regulatory sequence recognized by said transcriptional regulator protein, ligating the DNA and double-stranded oligonucleotide under conditions wherein at least two copies of the double-stranded oligonucleotide are incorporated at the restriction digestion site, and obtaining the recombinant promoter element.

23. A recombinant expression construct according to claim 1 wherein the transcriptional control element comprises a plurality of lac operator sequences.

24. A recombinant expression construct according to claim 23 wherein at least one of said lac operator sequences comprises an insertion, deletion or point mutation.

25. A construct according to claim 1, wherein the construct is Op4, Op8 or Op4-6.

26. A recombinant mammalian cell comprising the recombinant expression construct of claim 25.

27. A construct according to claim 8, further comprising a tissue-specific enhancer element.

28. A construct according to claim 27, wherein the tissue-specific enhancer element is a muscle-specific enhancer element.

29. A construct according to claim 8, wherein the promoter is a synthetic promoter.

30. A construct according to claim 8, wherein the promoter is a tissue-specific promoter.

31. A construct according to claim 30, wherein the tissue-specific promoter is a synthetic promoter.
